SEQUENCE LISTING (13 sequences)

SEQ ID NO: 1 - DNA, 63 bp, Artificial
  Synthesized Primer
ggccagtgaattgtaatacgactcactatagggaggcggtttttttttttttttttttttttt

SEQ ID NO: 2 - DNA, 22 bp, Phodopus sungorus
ggagattgacatcctccctgag

SEQ ID NO: 3 - DNA, 23 bp, Phodopus sungorus
gctctggatgtaccagctcttca

SEQ ID NO: 4 - DNA, 27 bp, Phodopus sungorus
ctggtgaataccccctctgtgcagctg

SEQ ID NO: 5 - DNA, 20 bp, Homo sapiens
acaccagtgctgcttcctga

SEQ ID NO: 6 - DNA, 22 bp, Homo sapiens
gagttttcgttgctgtcgtttg

SEQ ID NO: 7 - DNA, 29 bp, Homo sapiens
tttgtgtgtgaacccttgtttcctccaaa

SEQ ID NO: 8 - DNA, 20 bp, Homo sapiens
tggcagagaacggtgtgaag

SEQ ID NO: 9 - DNA, 18 bp, Homo sapiens
tggatgatgggcagagca

SEQ ID NO: 10 - DNA, 23 bp, Homo sapiens
catcacctccgtggccatgacca

SEQ ID NO: 11 - DNA, 20 bp, Rat
cggctaccacatccaaggaa

SEQ ID NO: 12 - DNA, 18 bp, Rat
gctggaattaccgcggct

SEQ ID NO: 13 - DNA, 22 bp, Rat
tgctggcaccagacttgccctc

(19) United States
(12) Patent Application Publication
(10) Pub. No.: US 2004/0106135 A1
(43) Pub. Date: Jun. 3, 2004

(54) Assay for PPAR ligand dependent gene modulation

(76) Inventors: Ranjan Mukherjee, Churchville, PA (US); Phillip Liu, Wilmington, DE (US); Reid M. Huber, Wilmington, DE (US); Timothy C. Burn, Hockessin, DE (US); Peter R. Young, San Francisco, CA (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/663,002

(22) Filed: Sep. 16, 2003

Related U.S. Application Data
(60) Provisional application No. 60/412,616, filed on Sep. 20, 2002.

(51) Int. Cl. C12Q 1/68

(52) U.S. Cl. 435/6

(57) ABSTRACT

The peroxisome proliferator activated receptor alpha (PPARα) plays a key role in mediating fatty acid metabolism by regulating expression of genes involved in fatty acid oxidation. A limitation of existing human cell models for testing PPARα ligands is the inability to detect PPAR responsive genes with endogenous levels of PPARα protein. The HK-2 cell line derived from human proximal tubules showed induction of several genes, including pyruvate dehydrogenase kinase 4 (PDK-4) and adipocyte differentiation related factor (ADRP) by PPARα ligands. Induction of PDK-4 by PPARα agonists in the HK-2 cell model closely correlates with its induction in vivo and thus represents a marker for PPARα agonist action. HK2 cells also exemplify the first model of a human cell line in which PPARα ligand dependent gene induction can be detected with endogenous levels of receptor.

The present patent application claims the benefit of U.S. Provisional Patent Application Serial No. 60/412,616, filed Sep. 20, 2002, the disclosure of which is incorporated herein by reference in its entirety.

### FIELD OF THE INVENTION

The present invention relates to the development of a human cell based assay for evaluating cellular responses to PPAR ligands. A limitation of existing cell models, such as HepG2 hepatoma cells, is the inability to detect PPAR responsive genes with endogenous levels of receptor expression. The present invention exemplifies the first model of a human cell line in which PPAR ligand dependent gene induction can be detected with endogenous levels of receptor.

### BACKGROUND OF THE INVENTION

The Peroxisome Proliferator Activated Receptor (PPAR) family of nuclear receptors is composed of three distinct genes PPARα, PPARγ and PPARδ(β) that play a central role in regulating the metabolism of lipids. All three receptor subtypes form a similar functional hetero-dimeric DNA binding complex with the 9-cis retinoic acid receptor (RXR), but due to structural variations in the ligand binding pocket, PPAR proteins are activated by distinct panels of ligands that lead to divergent pharmacological effects (Kliewer et al., (2001) Recent Prog Horm Res 56:239-263; Xu et al., (2001) Proc Natl Acad Sci U.S.A. 98:13919-13924). PPARγ binds preferentially to polyunsaturated fatty acids and the synthetic ligand class known as thiazolidinediones, which have been used for antidiabetic treatments. Ligands for PPARα are structurally diverse, including a variety of saturated fatty acids, xenobiotics and the fibrate class of hypolipidemic drugs (Kliewer et al., (1997) Proc Natl Acad Sci U.S.A. 94:4318-4323; Forman et al., (1997) Proc Natl Acad Sci U.S.A. 94:4312-4317.). Fibrates, such as gemfibrozil and fenofibrate, have been used clinically for the treatment of hyperlipidemia well before the identification of their molecular target PPARα. Specific binding of PPARα ligands to their cognate receptor was only demonstrated after the synthesis of newer ligands with significantly higher affinities. A role of PPARα protein in regulating lipid homeostasis in vivo was firmly established with genetic analysis of the PPARα null mouse. The null mice did not show decreases in serum lipid levels in response to fibrate treatment nor characteristic induction of peroxisomal enzymes involved in fatty acid oxidation (e et al., (1995) Mol. Cell. Biol. 15:3012-3022; Peters et al., (1997) J. Biol. Chem. 272:27307-27312). The molecular mechanism by which fibrates effect these physiological changes through PPARα is beginning to be understood by identification of PPARα target genes involved in lipoprotein metabolism. Global expression profiling of hepatic genes by DNA microarray analysis in animals treated with PPARα ligands revealed numerous targets that cluster into several functional pathways including mitochondrial, peroxisomal, and microsomal fatty acid oxidation illustrating the importance of PPARα in orchestrating the catabolism of lipids (Cherkaoui-Malki et al.,(2001) Gene Expr. 9:291-304; Yamazaki et al.,(2002) Biochem. Biophys. Res. Commun. 290:1114-1122). Although fibrates are effective in reducing serum lipids in many animal models, important differences exist between rodents and primates in their response to PPARα ligands. PPARα-dependent peroxisome proliferation and induction of genes encoding peroxisomal and microsomal enzymes involved in fatty acid oxidation is a characteristic response in rodent liver and primary hepatocytes. Moreover peroxisome proliferators induce pathological indications such as hepatomegaly and hyperplasia in the mouse and rat. These responses to PPARα activators are not evident in primary human hepatocytes, human hepatocarcinoma cell lines, or in liver biopsies of patients treated with fibrates (Willson et al., (2000) J. Med. Chem. 43:527-550). It has been hypothesized that these differences may be attributed to lower PPARα protein levels in human liver compared with rodent liver (Palmer et al., (1998) Mol. Pharmacol. 53:14-22; Gervois et al., (1999) Mol. Endocrinol. 13:1535-1549). However, HepG2 cells engineered to over-express PPARα protein still showed no induction of peroxisome proliferation-related genes in the human cells, suggesting that receptor expression is unlikely to be the cause for this species difference (Hsu et al.,(2001) J. Biol. Chem. 276:27950-27958; Lawrence et al.,(2001) J. Biol. Chem. 276:31521-31527). In addition, ligand binding and transactivation assays have revealed differences in the affinity by some PPARα ligands for rodent and human PPARα receptor affinities suggesting important variations in the ligand-binding domain that may dictate species-selective agonists (Mukheriee et al., (1994) J. Steroid Biochem. Mol. Biol. 51:157-166).

Collectively these differences underscore the need for developing human models for evaluating cellular responses to PPARα ligands. A limitation of the existing cell models, such as HepG2 hepatoma cells, is the inability to detect agonist-dependent responses with endogenous levels of receptor expression. PPARα over-expression cell lines have contributed to the identification of some PPAR target genes, but these cells also exhibit significantly higher basal levels of target gene expression that limit the magnitude of induction (Hsu et al.,(2001) J. Biol. Chem. 276:27950-27958; Lawrence et al.,(2001) J. Biol. Chem. 276:31521-31527). Thus the development of alternative cell models in which PPARα agonists could be evaluated using endogenous levels of receptor is needed.

Much effort has been directed toward understanding the role of PPARα in regulating gene expression and physiological responses in the liver however, little is known about the role of the receptor in extra-hepatic tissues. PPARα mRNA is strongly expressed in several metabolically active tissues involved in regulating fat and sugar storage and utilization including kidney, skeletal muscle, heart and pancreas (Mukherjee et al.,(1994) J. Steroid Biochem. Mol. Biol. 51:157-166). In one aspect of the present invention, in order, to address these questions and to develop alternative human cell models, an analysis of PPARα responsiveness in several human epithelial cell lines derived from tissues known to express PPARα mRNA or protein was undertaken.

Thus, in one aspect, the present invention comprises the characterization of a human proximal tubule-derived cell line (HK-2) that exhibits fibrate-dependent activation of PPARα target genes, including pyruvate dehydrogenase kinase-4 (PDK-4) and adipocyte differentiation related protein (ADRP). In another aspect of the present invention, it was observed that activation of PDK-4 correlates well between the HK-2 cells and its induction in vivo in hamster liver and kidney, notwithstanding differences in bioavailability in vivo. These results indicate that the transcriptional regulation of PDK-4 by PPARα in the HK-2 cells is a useful marker for assaying PPAR ligand activity. The present invention, therefore, addresses the need for a marker for assaying the cellular and in vivo response of a given PPAR ligand.

The publications and other materials referenced herein by author to illuminate the background of the invention or to provide additional details regarding the practice of the invention, are incorporated by reference in their entirety.

### SUMMARY OF THE INVENTION

The present invention provides a method of identifying a peroxisome proliferator activated receptor (PPAR) modulator. In one embodiment, the method comprises the steps of (a) determining a first level mRNA transcript of a PPAR responsive gene formed in a cell endogenously expressing one or more PPARs; (b) contacting the cell endogenously expressing the one or more PPARs with a test compound known or suspected to bind to the one or more PPARs; (c) measuring a second level of mRNA transcript of the PPAR responsive gene formed in the cell; and comparing the first level of mRNA transcript with the second level of mRNA transcript, wherein, a difference in the first and second levels of mRNA transcript indicates the test compound is a PPAR modulator. In some embodiments, the one or more PPARs is selected from the group consisting of PPAR-α, PPAR-β(δ), and PPAR-γ. In some embodiments, the cell is a mammalian cell, such as a human proximal tubule derived cell (HK-2). In yet other embodiments, the PPAR responsive gene is selected from the group consisting of pyruvate dehydrogenase kinase-4 (PDK-4) and adipocyte differentiation relating protein (ADRP).

The present invention also provides a method of identifying a peroxisome proliferator activated receptor (PPAR) modulator. In one embodiment, the method comprises the steps of (a) determining a first level of expression of a protein encoded by a PPAR responsive gene in a cell endogenously expressing one or more PPARs; (b) contacting the cell endogenously expressing the one or more PPARs with a test compound known or suspected to bind to the one or more PPARs; (c) measuring a second level of expression of the protein encoded by the PPAR responsive gene; and (d) comparing the second level of expression of the protein encoded by the PPAR responsive gene with the first level of protein encoded by the PPAR responsive gene, wherein, a difference in the first and second levels of expression of the protein encoded by the PPAR responsive gene indicates the test compound is a PPAR modulator. In a some embodiments, the one or more PPARs is selected from the group consisting of PPAR-α, PPAR-β(δ), and PPAR-γ. In some embodiments, the cell is a mammalian cell, such as a human proximal tubule derived cell (HK-2). In yet other embodiments, the PPAR responsive gene is selected from the group consisting of pyruvate dehydrogenase kinase-4 (PDK-4) and adipocyte differentiation relating protein (ADRP).

The present invention further provides a method of identifying a peroxisome proliferator activated receptor (PPAR) modulator. In one embodiment the method comprises the steps of (a) determining a baseline level of functional activity of a protein encoded by a PPAR responsive gene in a cell endogenously expressing one or more PPARs; (b) contacting the cell endogenously expressing the one or more PPARs with a test compound known or suspected to bind to the one or more PPARs; (c) measuring a post-contact level of functional activity of the protein encoded by the PPAR responsive gene; and (d) comparing the post-contact level of functional activity of the protein encoded by the PPAR responsive gene with the baseline level of functional activity of the protein encoded by the PPAR responsive gene, wherein, a difference in the first and second levels of functional activity of the protein encoded by the PPAR responsive gene indicates the test compound is a PPAR modulator. In some embodiments, the one or more PPARs is selected from the group consisting of PPAR-α, PPAR-β(δ), and PPAR-γ. In some embodiments, the cell is a mammalian cell, such as a human proximal tubule derived cell (HK-2). In yet other embodiments, the PPAR responsive gene is selected from the group consisting of pyruvate dehydrogenase kinase-4 (PDK-4) and adipocyte differentiation relating protein (ADRP). In yet further embodiments, the functional activity is selected from, but not limited to, the group consisting of an increase or decrease in kinase activity, an increase or decrease in insulin sensitization, and one or more changes in adipocyte differentiation.

Thus, it is an object of the present invention to provide a method of identifying a peroxisome proliferator activated receptor (PPAR) modulator. This object is achieved in whole or in part by the present invention.

Some of the objects of the invention having been stated hereinabove, other objects will become evident as the description proceeds when taken in connection with the accompanying drawings as best described hereinbelow.

### BRIEF DESCRIPTION OF THE FIGURES

FIGS. 1A and 1B depict transcriptional profiling of HK-2 cell response to GW9578 and indicates induction of PDK-4 and ADRP mRNA.

FIG. 1A is a plot depicting transcriptional profiling of HK-2 cell response to GW9578 and indicates induction of PDK-4 and ADRP mRNA. Changes in gene expression in HK-2 cells treated with DMSO or 300 nM GW9578 for 24 hours were profiled using DNA microarrays. Points corresponding to PDK-4 and ADRP are highlighted in the plot. Points falling outside of the inner lines are changed by greater than 2-fold; those falling outside the outer lines by greater than 3-fold.

FIG. 1B is a cluster analysis of transcriptional profiles for ACHN, HK-2, SW872 and HepG2 cells treated with GW9578. Threshold cutoffs were set at fold change >1.4; p<0.01; and statistically significant in at least 2 experiments. The genes fatty acid CoA ligase 2 (FACL2), palmitoyl acyl-CoA oxidase 1 (ACOX1), and carnitine palmitoyl acyl-CoA transferase (CPT1A) are included for comparison based on published reports that they are PPARα responsive genes in HepG2 cells over expressing PPARα (Hsu et al., (2001) J. Biol. Chem. 276:27950-8; Lawrence et al., (2001) J. Biol. Chem. 276:31521-7).

FIGS. 2A, 2B and 2C depict kinetics and dose-response curves for PDK-4 and ADRP mRNA induction in HK-2 cells by PPARα agonists.

FIG. 2A is a bar graph depicting time-course of induction of PDK4 and ADRP. HK-2 cells were treated with 100 nM GW9578 and harvested at the indicated times for RT-PCR analysis. The control sample (C) was treated with vehicle and harvested at 2 hours. Each bar represents the average and standard error of three samples.

FIG. 2B is a plot depicting the results of treating HK-2 cells with the indicated doses of GW9578, fenofibric acid or gemfibrozil for 24 hours and then analyzing for PDK-4 mRNA levels using RT-PCR. Values were normalized to 18S rRNA levels. The maximal induction by GW9578 was defined as 100%. Each point represents a single determination and shown is a representative experiment that was repeated with similar results.

FIG. 2C is a plot depicting the results of treating HK-2 cells with the indicated doses of GW9578, fenofibric acid or gemfibrozil for 24 hours and then analyzing for ADRP mRNA levels using RT-PCR. Values were normalized to 18S rRNA levels. The maximal induction by GW9578 was defined as 100%. Each point represents a single determination and shown is a representative experiment that was repeated with similar results.

FIG. 3 depicts PPARα and RXRα protein levels in human cell lines. Nuclear extracts were prepared from the indicated cell lines, and 10 μg of each extract was subjected to SDS-PAGE followed by immunoblotting using antibodies specific for PPARα (upper panel) or RXRα (lower panel).

FIG. 4A is a bar graph depicting the induction of PDK-4 mRNA in hamster liver by PPARα ligands. Fat-fed hamsters were given one dose of GW9578 (3 mg/kg), fenofibrate (100 mg/kg) or gemfibrozil (500 mg/kg) or methocel (vehicle). After 16 hours, the tissues were harvested, total RNA isolated and levels of hamster PDK-4 mRNA were detected by RT-PCR. The values are normalized to 18S rRNA levels. The mean and SEM of three individual animals are shown.

FIG. 4B is a bar graphs depicting the induction of PDK-4 mRNA in hamster kidney by PPARα ligands. Fat-fed hamsters were given one dose of GW9578 (3 mg/kg), fenofibrate (100 mg/kg) or gemfibrozil (500 mg/kg) or methocel (vehicle). After 16 hours, the tissues were harvested, total RNA isolated and levels of hamster PDK-4 mRNA were detected by RT-PCR. The values are normalized to 18S rRNA levels. The mean and SEM of three individual animals are shown.

FIG. 5 is a bar graph depicting PDK4 induction by ligands selective for different PPARs.

### DETAILED DESCRIPTION

PPARα ligands such as fibrate drugs are important therapeutic compounds in the treatment of hyperlipidemia in humans. Because important differences exist among species in their pathophysiological responses and in ligand affinities for PPARα, there is a need for developing human cell models for studying PPARα activity. In one aspect of the present invention, the PPARα responsiveness of a human cell line HK-2 derived from proximal tubule cells isolated from a normal kidney (Ryan et al., (1994) Kidney Int. 45:48-57) is characterized. HK-2 cells represent the first reported human cell line that shows detectable induction of PPARα responsive genes with endogenous levels of PPARα protein. In one aspect of the present invention, using this model, two PPARα responsive genes that are significantly induced by PPARα ligands have been identified, namely PDK-4 and ADRP. The rank order potency for the three fibrates tested (ureido-thioisobutyric acid (GW9578), gemfibrozil and fenofibric acid) is the same for both genes; however, the EC₅₀ values for PDK-4 are lower than ADRP for each ligand. As a model, PDK-4 induction in HK-2 cells closely paralleled the responses to fibrates in the fat-fed hamster liver suggesting that the cell based assay might be a good surrogate for the in vivo action of these drugs.

Several lines of evidence reinforce the position that the observed effects reported in the present disclosure are indeed bona fide PPARα responses. First both PDK-4 and ADRP are induced within 2 hours in a dose-dependent manner by three characterized PPARα ligands, GW9578, fenofibric acid and gemfibrozil, with a rank-order potency that mirrors their activity in cell-based transactivation assays (Willson et al., (2000) J. Med. Chem. 43:527-550; Mukherjee et al., (2002) J. Steroid Biochem. Mol. Biol. 1712:1-9). For PDK-4 induction EC₅₀ values of 0.01, 10 and 27 μM are observed for GW9578, fenofibric acid and gemfibrozil which closely match the published values for these drugs by GAL4-hPPARα (see Table 1, and references therein).

TABLE 1

| | | EC₅₀ (μM) in Transactivation Assay | | |
|---|---|---|---|---|
| | | (GAL4-hPPARα) | | |
| | | | Ref | Ref |
| | | | (Mukherjee et al., | (Willson et |
| | | Present | (2002) J. Steroid | al., (2000) J. |
| | EC₅₀ (μM) in | Dis- | Biochem. Mol. | Med. Chem. |
| Compounds | HK-2 (PDK-4) | closure | Bio 1712: 1-9) | 43: 527-550) |
| GW9578 | 0.01 | 0.01 | | 0.05 |
| Fenofibric | 10 | 80 | 30 | 30 |
| Acid | | | | |
| Gemfibrozil | 27 | 60 | 59 | |

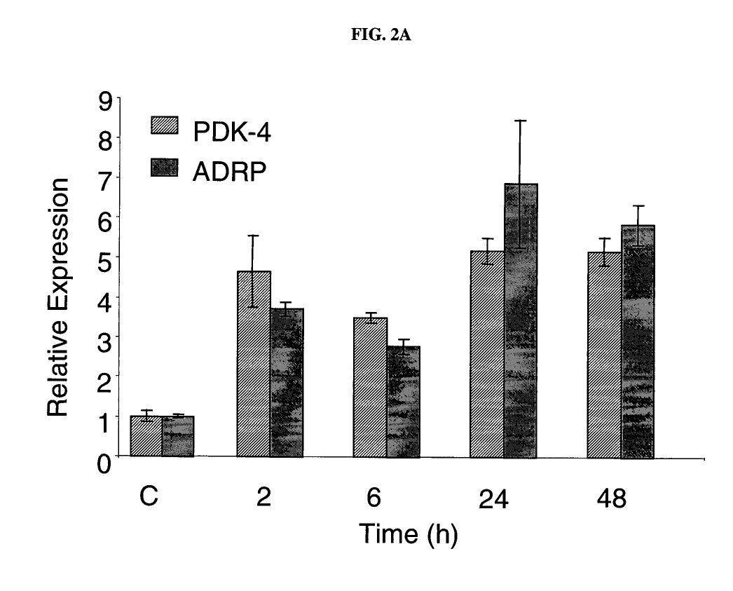

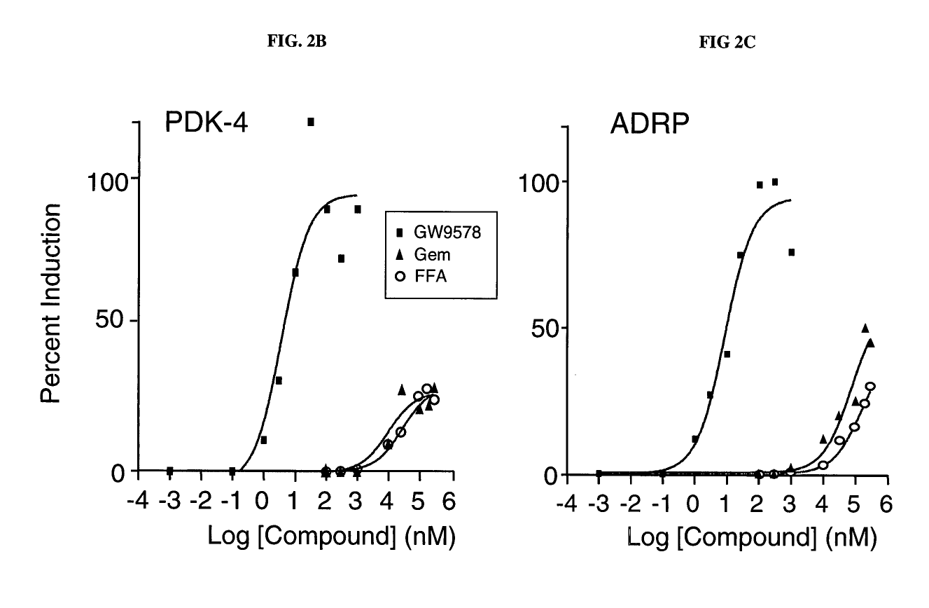

Table 1 depicts a comparison of PPARα agonist activity between an endogenous target in HK-2 cells and a reporter gene in transactivation assays. EC₅₀ values were determined for PDK-4 mRNA induction by quantitative PCR under conditions used to generate the data shown in FIG. 2. EC₅₀ values in the transactivation assays were determined by transfection of an expression plasmid encoding a GAL4-hPPARα chimera into cells co-expressing a luciferase reporter gene under the transcriptional control of GAL4 upstream activating elements.

The selectivity of GW9578 and fenofibric acid is 10-20 fold for PPARα versus PPARγ (Willson et al., (2000) J. Med. Chem. 43:527-550), indicating that the induction of PDK-4 at these doses is through PPARa. In vivo, PDK-4 induction was observed in rodent tissues following exposure to another PPARα agonist, WY-14, 643 (Wu et al.,(1999) Diabetes 48:1593-1599), and these responses were abrogated in PPARα−/−mice (Sugden et al., (2001) Arch. Biochem. Biophys. 395:246-252; Wu et al., (2001) Biochem. Biophys. Res. Commun. 287:391-396) adding further support to the contention that PDK-4 is a PPARA target gene.

The observation that human PDK-4 is induced by fibrates is intriguing for several reasons, for example because PDK-4, together with other PDK isozymes, regulates a key step in oxidative glucose metabolism by catalyzing the phosphorylation and inactivation of the mitochondrial pyruvate dehydrogenase complex (PDC). PDK-4 expression and activity increases, thus decreasing PDC activity, during periods of starvation when glucose sparing is needed (Wu et al.,(1998) Biochem. J. 329(1): 197-201). PDK-4 mRNA levels are also elevated in the heart of diabetic rats and in the muscle of high fat fed rats (Wu et al.,(1998) Biochem. J. 329(1): 197-201; Holness et al., (2000) Diabetes 49:775-781). Moreover, in a rat hepatoma cell line, PDK-4 mRNA was increased by free fatty acids an effect that was partially antagonized by insulin (Huang et al., (2002) Diabetes 51:276-283). The present disclosure that PDK-4 is induced within 2 hours in HK-2 cells in a dose-dependent manner to fibrates with a rank-order potency that follows the binding affinities for the receptor corroborates the genetic evidence that PDK-4 is a PPARα target gene (Sugden et al., (2001) Arch. Biochem. Biophys. 395:246-252; Wu et al., (2001) Biochem. Biophys. Res. Commun. 287:391-396), and, demonstrates that PDK-4 is a conserved PPARα target in human cells as well as in rodents. In obese humans, increased PDK activity has been associated with insulin resistance and non-insulin dependent diabetes mellitus (Majer et al., (1998) Mol. Genet. Metab. 65:181-186). In addition, PDK activity and PDK-4 mRNA and protein levels are increased in human skeletal muscle in subjects on a high fat/low carbohydrate diet (Peters et al., (2001) Am. J. Physiol. Endocrinol. Metab. 281: E1151-1158). The PPARγ ligand GW1929 has been shown to decrease PDK-4 levels in the skeletal muscle of rats, an effect that might be subsequent to a decrease in FFA (Way et al., (2001) Endocrinol. 142:1269-1277). Although it is not the inventors' desire to be bound to any theory of operation, based upon these results, it is possible that the increase in PDK-4 levels in vivo would be transient, followed by a decrease when FFA levels are reduced by fibrate administration.

The HK-2 cell model provides an attractive alternative to receptor and reporter over-expression cell lines for screening PPARα agonists because both the receptor and its target genes are in their native chromatin context. Using HK-2 cells for evaluation of PPARα transcriptional responses also avoids the problem of increased basal levels of gene expression associated with PPARα over-expressing cell lines which decreases the fold induction in response to exogenous ligands (Hsu et al., (2001) J. Biol. Chem. 276:27950-27958; Lawrence et al., (2001) J. Biol. Chem. 276:31521-31527). Both HK-2 and SW872 cell express detectable levels of PPARα protein in their nuclei, and in contrast to prior reports, PPARa protein was also detected in HepG2 cells. The levels of PPARα in the three cell lines varied only modestly indicating that weak PPARα activity in HepG2 cells cannot be attributable only to low levels of the receptor. Consistent with results from the over-expression cell lines, as reported herein small increases in the ECH1 and ACAA2 genes were also observed, with no induction of peroxisomal proliferation-related genes such as the peroxisomal fatty acid-CoA oxidase, thiolase or enoyl-CoA hydratase.

The physiological importance of PPARα in extra-hepatic tissues has not been examined in great detail. Kidney is an organ that expresses high levels of PPARα (Mukherjee et al., (1997) J. Biol. Chem. 272:8071-8076) and renal epithelia depend upon fatty acid oxidation for energy (Wirthensohn & Guder, (1983) Miner. Electrolyte Metab. 9:203-211). Therefore PPARα is likely to be involved in regulating fatty acid oxidation and energy generation in this tissue. Interestingly glucocorticoids increased expression of PPARα in rat kidney, and dexamethasone together with oleic acid (a PPARα ligand) induced mRNA levels of medium chain acyl CoA dehydrogenase in a transformed primary renal cell line from rabbit cortical epithelium (Diouadi & Bastin, (2001) J. Am. Soc. Nephrol. 12:1197-1203).

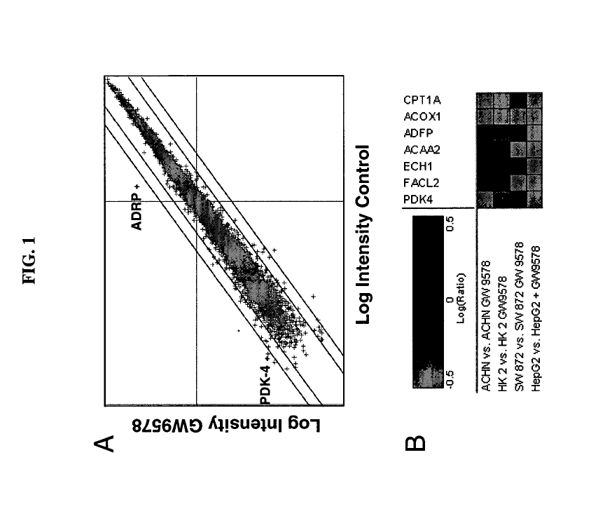

In another aspect of the present invention, the gene expression profiles of several human epithelial cell lines were surveyed upon treatment with the PPARα agonist GW9578 by DNA microarray analysis. Cell lines were chosen based upon information suggesting PPARα expression or responsiveness to agonists in these lines (SW872, LNCaP) or in the corresponding tissues in animal models (e.g. kidney for ACHN and HK-2) (Horoszewicz et al., (1983) Cancer Res. 43:1809-1818; Jiang et al., (2001) J. Lipid Res. 42:716-724; Ryan et al., (1994) Kidney Int. 45:48-57; Mukherjee et al., (1997) J. Biol. Chem. 272:8071-8076). For comparison, a standard cell model for PPARa analysis was also profiled, the human hepatoma cell line HepG2. In the renal cell line HK-2 and the preadipocyte line SW872 there were several target genes that were significantly induced (>2-fold, p<0.01) by GW9578 when compared with cells treated with vehicle (FIG. 1A). The two transcripts that are the most highly induced encode adipocyte differentiation related protein (ADRP) and pyruvate dehydrogenase kinase (PDK-4). Both ADRP and PDK-4 were increased approximately 4 fold in GW9578-treated HK-2 cells compared with controls. ADRP was also significantly induced in ACHN cells. A number of transcripts were significantly changed (1.4-2-fold change, P<0.01) in different microarray experiments. To ascertain whether these genes were responsive to GW9578 in multiple cell lines, a cluster analysis across all data sets was performed. This analysis revealed that two additional genes ECH1 and ACAA2 were significantly induced in at least two cell lines (FIG. 1B). Previous studies using HepG2 cells that over-expressed PPARα revealed significant increases in mitochondrial camitine palmitoyl acyl CoA transferase (CPT1A), palmitoyl acyl-CoA oxidase 1 (ACOX1), and fatty-acid-CoA ligase, long-chain 2 (FACL2) upon PPARα agonist exposure (Lawrence et al., (2001) J. Biol. Chem. 276:31521-31527). However, only minor changes in FACL2 and CPT1 levels were observed in single experiments and no induction of ACOX1 was observed in any cell line tested. No response in any of these genes in HepG2 cells was observed with endogenous levels of PPARα. Similarly, there were only minor changes in gene expression in Caki-1 and LNCaP cells.

In another aspect of the present invention, in order to quantify the changes in expression more precisely and to confirm the microarray results, real-time PCR assays for quantitative analysis of human ADRP and PDK-4 mRNA levels were developed. In this aspect of the present invention, using quantitative PCR assays a time-course of induction by GW9578 was prepared. The kinetics of mRNA induction was rapid for both genes with near maximal increases occurring by 2 hours (FIG. 2A).

In order to compare the rank-order potency of three well-characterized PPARα agonists, dose-response curves were generated for the induction of both genes. A representative example of the dose-response curves in HK-2 cells is shown in FIG. 2B.

GW9578 was the most potent activator of PDK-4 with an EC₅₀ of approximately 10 nM. The responses to fenofibric acid and gemfibrozil were nearly equivalent with a lower efficacy than GW9578 and EC₅₀ values of 10 and 27 μM, respectively. The magnitude of PDK-4 induction varied from approximately 3 to 7-fold among experiments depending upon the basal level of PDK-4 expression that correlated with cell-density, but neither the EC₅₀ values nor the rank-order of potency by the three compounds was altered. The induction of ADRP by these drugs followed the same rank-order potency for PDK-4 though the EC₅₀ values were slightly higher (FIG. 2C). For both gemfibrozil and fenofibric acid, the induction of ADRP did not appear to saturate even at the highest dose tested (300 μM), but solubility limitations precluded testing higher concentrations. The rank-order potency, as well as the activity of these compounds in the HK-2 cell model closely tracks their activity in a cell-based transactivation assay using a GAL4-hPPARα chimera (Table 1, presented herein). Indeed, the EC₅₀ values obtained for GW9578 and gemfibrozil using these two methods were nearly equivalent, supporting the position that induction of PDK-4 and ADRP in HK-2 cells is acting through PPARα.

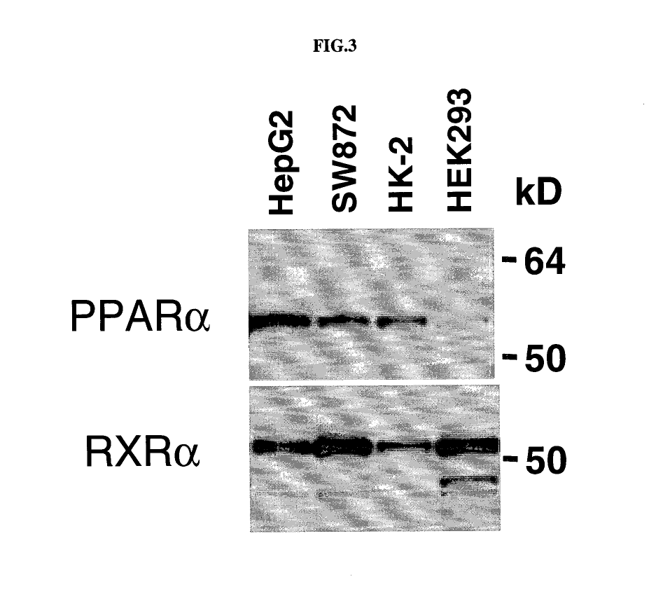

In yet a further aspect of the present invention, in order to determine the endogenous levels of PPARα protein in the different cell lines that were profiled by DNA microarrays, immunoblotting was performed. Because endogenous levels of PPARα are low, being undetectable in whole cell extracts from liver and hepatoma cells (Palmer et al., (1998) Mol. Pharmacol. 53:14-22; Hsu et al., (2001) J. Biol. Chem. 276:27950-27958), nuclear extracts were used in order to concentrate the PPARa signal. As shown in FIG. 3, PPARα protein was detectable in HK-2, SW872 as well as in HepG2 extracts. In contrast, another human kidney cell line HEK293 cells expresses little or no PPARα. The dimerization partner of PPARα, RXRα was also detected in nuclear extracts from all of the cells.

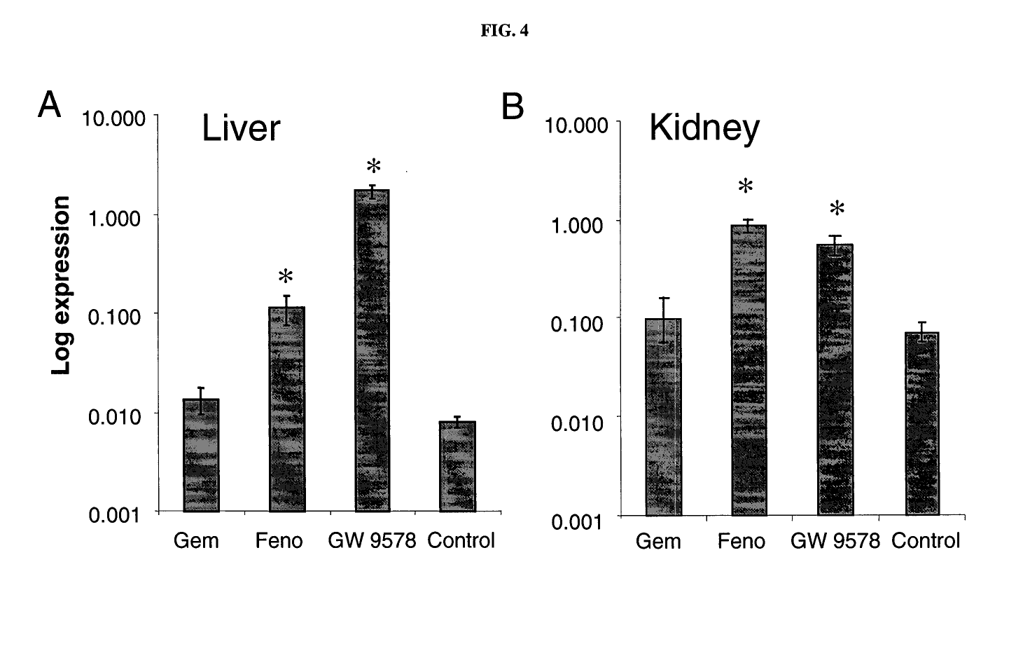

PDK-4 plays a critical role in regulating glucose metabolism by phosphorylating and inactivating the pyruvate dehydrogenase complex in response to increased fatty acid oxidation. Recently it was demonstrated that PPARα ligands induced PDK-4 expression in the kidney of wild-type mice, but not in PPARα null mice (Sugden et al., (2001) Arch. Biochem. Biophys. 395:246-252; Wu et al., (2001) Biochem. Biophys. Res. Commun. 287:391-396). To investigate the response of PDK-4 to fibrate treatment in vivo, in another aspect of the present invention the fat-fed hamster model was employed, which closely mimics the serum lipid profiles of humans than other rodent models (Sullivan et al., (1993) Lab. Anim. Sci. 43:575-578). To develop the in vivo model, hamsters were kept on a high cholesterol diet for 5 days prior to a single dose of GW9578 (3 mg/kg), fenofibrate (100 mg/kg), gemfibrozil (500 mg/kg), or methocel control. These doses were previously shown to yield maximal effects on blood triglycerides in hamster. Using an RT-PCR assay for the hamster PDK-4 mRNA, a dramatic induction of PDK-4 levels by GW9578 (200-fold) and a modest increase by fenofibrate (14-fold) in the liver at 16 hour post-treatment was detected, whereas gemfibrozil had the least effect. Gemfibrozil also had the lowest activity in a hamster PPARα transactivation assay. In hamster kidney, the basal levels of PDK-4 mRNA were approximately 10-fold higher than in the liver (FIG. 4B). Thus the relative increase in expression by fibrate treatment was reduced compared to the liver; however, both GW9578 and fenofibrate significantly induced PDK-4 (7 and 11-fold, respectively) while gemfibrozil had no effect. Fibrates are effective treatments for lowering serum triglyceride levels. To verify that the doses used in this study were efficacious, blood triglyceride levels were monitored over a 48 hour time course. As shown in Table 2 (presented herein), GW9578 significantly decreased triglyceride levels compared with the control group at all three time points while neither gemfibrozil nor fenofibrate altered triglyceride levels to a statistically significant degree at this early time point.

TABLE 2

| Treatment | 8 h | 16 h | 48 h |
|---|---|---|---|
| Control | 261.33 ± 27.33 | 308.00 + 33.13 | 208.67 + 27.72 |
| (methocel) | | | |
| Gemfibrozil | 334.33 + 82.42 | 325.67 + 46.77 | 206.33 + 65.30 |
| Fenofibrate | 229.00 + 38.52 | 231.67 + 63.22 | 146.33 + 17.15 |
| GW9578 | 106.00 + 46.22* | 142.33 + 51.31* | 104.67 + 12.02* |

Table 2 summarizes serum triglyceride levels observed in fat fed hamsters dosed with PPARα ligands. Fat-fed hamsters were treated with GW9578 (3 mg/kg), fenofibrate (100 mg/kg) or gemfibrozil (500 mg/kg) or methocel control. Blood was sampled at 8, 16 and 48 hours following a single drug treatment for total triglyceride analysis. The mean levels (+/−SEM) for 3 animals per group are shown. The “*” indicates a statistically significant difference with respect to control (p<0.01) by Student's t-test.

In a separate longer-term study, all three compounds significantly reduced serum triglycerides at 10 days post treatment at the same doses used here. Therefore, these data indicate that the rapid transcriptional response of PDK-4 in hamster liver and kidney correlates with physiologically relevant endpoints of PPAR agonist action and is a useful surrogate for assaying the effectiveness of PPAR ligands.

In summary, characterization of cell models like HK-2 cells can foster further understanding of the activity of human PPARα under conditions where both the receptor and its targets are in their native chromatin context. Further, the correlation between PDK-4 induction in HK-2 cells by PPARα ligands and their effect in vivo on both PDK-4 mRNA levels and triglyceride lowering indicates that this assay is a valuable assay that facilitates the rapid analysis of PPARα ligand-binding activity in a human cell line.

The present invention is further detailed in the following Examples, which are offered by way of illustration alone and are not intended to limit the invention in any manner. Standard techniques well known to those skilled in the art, or the techniques specifically described below, are utilized.

### EXAMPLES

Chemicals: Gemfibrozil and fenofibrate were purchased from Sigma Chemical Co., fenofibric acid and GW9578 (Brown et al., J. Med. Chem. (1999) 42:3785-3788) were synthesized.

Cell culture: The following cell lines were obtained from American Type Culture Collection (ATCC) and cultured in the recommended medium for each cell type: HepG2, HK-2, Caki-1, LNCaP (CloneFGC), SW872, and ACHN. PPARα compounds were prepared to a 500× stock in DMSO; corresponding control cells received an equal volume of vehicle (0.2% v/v).

### Example 1

RNA isolation: Total RNA isolations were performed using the RNeasy total RNA isolation system and DNased according to the manufacturer's (Qiagen) instructions. RNA purity and concentration was determined spectrophotometrically (260 nm/280 nm); integrity was assessed by agarose gel electrophoresis.

### Example 2

Expression profiling: Expression profiling of RNA samples was performed essentially as described (Lockhart et al., (1996) Nat. Biotechnol. 14:1675-80) using the Affymetrix human U95Av2 array. Briefly, RNA was isolated from 100 mm cell culture dishes, DNased, and 15 μg used as a template for double stranded cDNA synthesis according to standard protocols (Invitrogen). Reverse transcription was primed using a T7-modified oligo-dT primer (5-GGCCAGTGAATTGTAATACGACTCACTATAGGGAGGCGG-d(T)₂₄-3′) (SEQ ID NO: 1). In vitro transcription was then performed on each double stranded cDNA synthesis, according to the manufacturer's (Enzo Diagnostics) instructions. The resultant cRNA products were purified using RNeasy columns (Qiagen), pooled, and quantitated spectrophotometrically. For each sample, 20 μg of in vitro transcribed cRNA was fragmented by heating the sample to 94° C. in the presence of 40 mM Tris-acetate, pH 8.1, 100 mM potassium acetate, and 30 mM magnesium acetate. Hybridization cocktails contained 0.05 μg/μL fragmented cRNA, 50 pM control B2 oligonucleotide, 1.5, 5, 25, 100 pM of BioB, BioC, BioD, and Cre spiked cRNAs, 0.1 mg/mil herring sperm DNA, 0.5 mg/mL acetylated BSA, 100 mM MES, 1M NaCl, 20 mM EDTA, and 0.01% Tween-20. Hybridization, washing, and scanning were performed according to the manufacturer's (Affymetrix) recommendations. Image acquisition and segmentation were performed using GeneChip 4.0 (Affymetrix) according to the manufacturer's instructions. Affymetrix CEL files containing all raw data were exported for downstream analysis.

### Example 3

Data Analysis: Affymetrix CEL files from GeneChip 4.0 (Affymetrix) were imported into Resolver 2.0 under an empirically derived Affymetrix error model (Rosetta Inpharmatics). This error model is based on a series of control hybridizations that allow for the determination of the inherent variability within the Affymetrix system, and the identification of raw data parameters associated with that variability. Accordingly, the statistical significance (P-value) of a given expression data point takes into account the underlying error associated with the Affymetrix transcript abundance measurements as determined by this platform-specific error model. The null hypothesis for this P-value is that the transcript has a unity expression ratio. Clustering analysis was performed using an agglomerative hierarchical clustering algorithm where error-weighted log(ratio) correlation coefficients are used as similarity measurements (Hartigan, (1975) Clustering Algorithms, John Wiley & Sons, New York). Profile correlation analyses were performed using an X-Y plotting algorithm taking into account both transcript log(ratio) expression changes and the underlying error associated with each measurement.

### Example 4

Real-time PCR: Fluorescence-based real-time PCR was performed essentially as described (Abbaszade et al., (1999) J. Biol. Chem. 274:23443-23450). Primers and probes designed from the hamster pyruvate dehydrogenase kinase 4 (PDK4) sequence (Genbank Accession No. AF321218), human pyruvate dehydrogenase kinase 4 (PDK-4) sequence (Genbank Accession No. NM_{—}002612) and human adipose differentiation-related protein (ADFP) sequence (Genbank Accession No. NM_{—}001122) were synthesized and purified by Biosearch Technologies. Probes for ADFP and PDK-4 were modified at the 5′ end with the reporter dye 6-FAM, and at the 3′ end with the quencher dye Black Hole Quencher 1 (Biosearch Technologies). Probes detecting rat 18S rRNA were modified at the 5′ end with VIC and at the 3′ end with TAMRA (Biosearch Technologies). For detection of hamster PDK4, primers 5′-GGAGATTGACATCCTCCCTGAG (SEQ ID NO:2), 5′GCTCTGGATGTACCAGCTCTTCA (SEQ ID NO:3), and probe 5′-CTGGTGAATACCCCCTCTGTGCAGCTG (SEQ ID NO:4) were used. For detection of human PDK4, primers 5′-ACACCAGTGCTGCTTCCTGA (SEQ ID NO:5), 5′-GAGTTTTCGTTGCTGTCGTTTG (SEQ ID NO:6), and probe 5′-TTTGTGTGTGAACCCTTGTTTCCTCCAAA (SEQ ID NO:7) were used. For detection of human ADFP, primers 5′-TGGCAGAGAACGGTGTGAAG (SEQ ID NO:8), 5′-TGGATGATGGGCAGAGCA (SEQ ID NO:9), and probe 5′-CATCACCTCCGTGGCCATGACCA (SEQ ID NO: 10) were used. For detection of 18s rRNA, primers 5′-CGGCTACCACATCCAAGGAA (SEQ ID NO:11), 5′-GCTGGAATTACCGCGGCT (SEQ ID NO:12), and probe 5′-TGCTGGCACCAGACTTGCCCTC (SEQ ID NO:13) were used. Template cDNA was generated using the Advantage RT-PCR kit according to the manufacturer's (Clontech) instructions using random hexamers and 1 μg of DNaseI-treated total RNA. Taqman-based real-time PCR expression profiling was performed using 25 ng of each cDNA according to the manufacturer's (PE Biosystems) instructions with fluorescence being monitored in real-time with an ABI Prism 7700 (PE Biosystems). Relative expression levels were determined essentially as described (Gibson et al., (1996) Genome Res. 6:995-1001) using standard curves for each transcript. Relative abundance was then determined from these standard curves, subtracting mRNA levels obtained from negative control reactions performed in the absence of reverse transcriptase, and normalized to 18S rRNA levels. All expression measurements were performed in duplicate in two independent assays, generating a total of four measurements per cDNA.

### Example 5

Nuclear Protein Isolation and Immunoblotting: Nuclear proteins were isolated from cell lines by the method of Dignam (Dignam, (1990) Methods Enzymol. 182:194-203). Contamination by unbroken cells was determined by staining the nuclear pellet with Trypan Blue and estimated to be less than 5%. Protein concentration was estimated using the Bradford reagent. Equal amounts of nuclear protein (10 μg) were resolved by SDS-PAGE, transferred to PVDF membranes, incubated with anti-PPARα (Geneka Biotechnology) or RXRα (Santa Cruz Biotechnology) antiserum according to manufacturer's instructions followed by appropriate secondary antibodies and developed with enhanced chemiluminescent reagents.

### Example 6

Transcriptional activation assay: Cell based transcriptional activation assays using a GAL4-hPPARa expression plasmid and an HEK293 cell line stably integrated with a GAL4 UAS-Luciferase reporter gene was performed exactly as described previously (Mukherjee et al., (2002) J. Steroid Biochem. Mol. Biol. 1712:1-9).

### Example 7

Animal studies: All procedures performed in this study were approved by the Animal Care and Use committee, and conform to the guide for the Care and Use of Laboratory Animals Act. Male Syrian Golden hamsters (Charles River, Wilmington Mass.) weighing 120-140 g, were used in the study. Animals were kept on a 12-hour light/dark cycle and allowed free access to normal chow and water. The animals were divided into groups according to weight, and fed either an high fat diet (0.5% cholesterol, 5% coconut oil) (N=3) or normal chow diet (N=3). After 5 days on the diet the hamsters were dosed with 0.4 μL of compound with vehicle (0.5% methocell)

Compounds evaluated were Fenofibrate at 100 mg/kg Gemfibrozil at 500 mg/kg and GW9578 at 3 mg/kg. At intervals of 0, 8, 16 and 48 hr animals were anesthetized with CO₂. Blood was collected by cardiac puncture into EDTA containing tubes; plasma was isolated for immediate analysis of triglycerides by the Dade Clinical Analyser®. Animals were then euthanized with CO₂. The liver and kidneys were collected, flash frozen in liquid nitrogen and stored at −80° C. for future gene expression analysis.

### Example 8

HEK293 cells were treated for 4 hours with the following PPAR modulators:

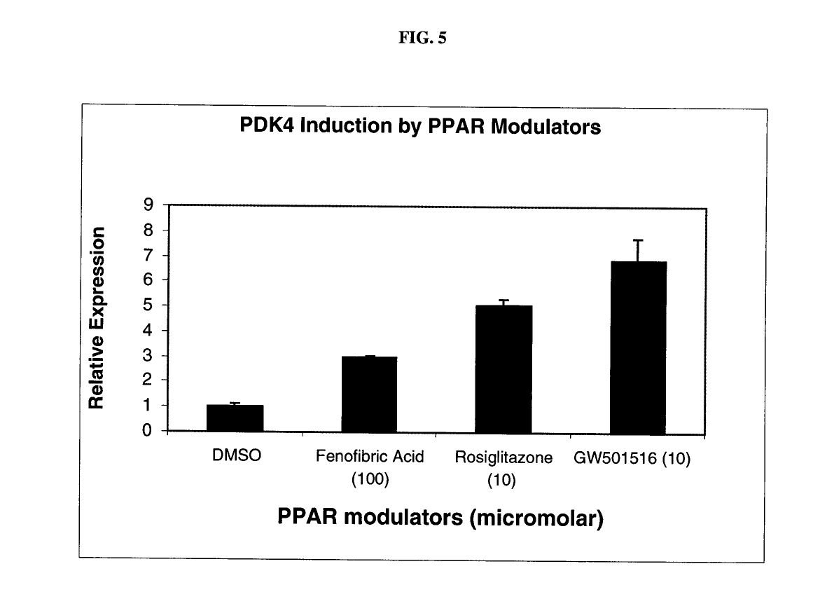

100 micromolar fenofibric acid (a PPARα selective modulator), 10 micromolar rosiglitazone (a PPARγ selective modulator) and 10 micromolar GW501516 (a PPARβ(δ) selective modulator; Oliver et al., (2001) Proc. Natl. Acad. Sci. U.S.A. 98:5306-11). PDK4 mRNA was measured by Taqman PCR analysis. The PDK4 expression levels are expressed relative to CYCD1 mRNA used as a normalization control. The results of this experiment are presented in FIG. 5. The figure indicates that PDK4 is induced by modulators of a variety of PPAR isoforms.

While the invention has been disclosed in this patent application by reference to the details of preferred embodiments of the invention, it is to be understood that the disclosure is intended in an illustrative rather than in a limiting sense, as it is contemplated that modifications will readily occur to those skilled in the art, within the spirit of the invention and the scope of the appended claims.

What is claimed is:

1. A method of identifying a peroxisome proliferator activated receptor (PPAR) modulator comprising the steps of:
(a) determining a first level mRNA transcript of a PPAR responsive gene formed in a cell endogenously expressing one or more PPARs;
(b) contacting the cell endogenously expressing the one or more PPARs with a test compound known or suspected to bind to the one or more PPARs;
(c) measuring a second level of mRNA transcript of the PPAR responsive gene formed in the cell; and
(d) comparing the first level of mRNA transcript with the second level of mRNA transcript, wherein, a difference in the first and second levels of mRNA transcript indicates the test compound is a PPAR modulator.

2. The method of claim 1, wherein the one or more PPARs is selected from the group consisting of PPAR-α, PPAR-β(δ), and PPAR-γ.

3. The method of claim 1, wherein the cell is a mammalian cell.

4. The method of claim 3, wherein the mammalian cell is a human proximal tubule derived cell (HK-2).

5. The method of claim 1, wherein the PPAR responsive gene is selected from the group consisting of pyruvate dehydrogenase kinase-4 (PDK-4) and adipocyte differentiation relating protein (ADRP).

6. A method of identifying a peroxisome proliferator activated receptor (PPAR) modulator comprising the steps of:
(a) determining a first level of expression of a protein encoded by a PPAR responsive gene in a cell endogenously expressing one or more PPARs;
(b) contacting the cell endogenously expressing the one or more PPARs with a test compound known or suspected to bind to the one or more PPARs;
(c) measuring a second level of expression of the protein encoded by the PPAR responsive gene; and
(e) comparing the second level of expression of the protein encoded by the PPAR responsive gene with the first level of protein encoded by the PPAR responsive gene, wherein, a difference in the first and second levels of expression of the protein encoded by the PPAR responsive gene indicates the test compound is a PPAR modulator.

7. The method of claim 6, wherein the one or more PPARs is selected from the group consisting of PPAR-α, PPARβ(δ), and PPAR-γ.

8. The method of claim 6, wherein the cell is a mammalian cell.

9. The method of claim 8, wherein the mammalian cell is a human proximal tubule derived cell (HK-2).

10. The method of claim 8, wherein the PPAR responsive gene is selected from the group consisting of pyruvate dehydrogenase kinase-4 (PDK-4) and adipocyte differentiation relating protein (ADRP).

11. A method of identifying a peroxisome proliferator activated receptor (PPAR) modulator comprising the steps of:
(a) determining a baseline level of functional activity of a protein encoded by a PPAR responsive gene in a cell endogenously expressing one or more PPARs;
(b) contacting the cell endogenously expressing the one or more PPARs with a test compound known or suspected to bind to the one or more PPARs;
(c) measuring a post-contact level of functional activity of the protein encoded by the PPAR responsive gene; and
(f) comparing the post-contact level of functional activity of the protein encoded by the PPAR responsive gene with the baseline level of functional activity of the protein encoded by the PPAR responsive gene, wherein, a difference in the first and second levels of functional activity of the protein encoded by the PPAR responsive gene indicates the test compound is a PPAR modulator.

12. The method of claim 11, wherein the one or more PPARs is selected from the group consisting of PPAR-α, PPAR-β(δ), and PPAR-γ.

13. The method of claim 11, wherein the cell is a mammalian cell.

14. The method of claim 13, wherein the mammalian cell is a human proximal tubule derived cell (HK-2).

15. The method of claim 11, wherein the PPAR responsive gene is selected from the group consisting of pyruvate dehydrogenase kinase-4 (PDK-4) and adipocyte differentiation relating protein (ADRP).

16. The method of claim 11, wherein the functional activity is selected from the group consisting of an increase or decrease in kinase activity, an increase or decrase in insulin sensitization, and one or more changes in adipocyte differentiation.
